(19) United States
(12) Patent Application Publication
(10) Pub. No.: US 2013/0072802 A1
(43) Pub. Date: Mar. 21, 2013

(54) Lens for Ultrasonic Diagnosis Apparatus and Probe for Ultrasonic Diagnosis Apparatus

(71) Applicant: Satoshi Hozumi, Kodama-gun (JP)

(72) Inventor: Satoshi Hozumi, Kodama-gun (JP)

(73) Assignee: SHIN-ETSU POLYMER CO., LTD., Tokyo (JP)

(21) Appl. No.: 13/700,529

(22) PCT Filed: May 25, 2011
(86) PCT No.: PCT/JP2011/002898

§ 371 Date: Nov. 28, 2012

(30) Foreign Application Priority Data
May 31, 2010 (JP) 2010-124380

(51) Int. Cl. A61B 8/00

(52) U.S. Cl. CPC A61B 8/4281; A61B 8/4444
USPC 600/472

(57) ABSTRACT

This invention provides a lens for ultrasonic diagnosis apparatuses having requisite acoustic characteristics concerning sonic velocity, acoustic impedance and acoustic attenuation and exhibiting high abrasion resistance; the lens including a base formed from a silicone rubber composition, and a urethane coat layer placed on an outer surface of the base, wherein the urethane coat layer includes 100 parts by mass of a urethane resin and 3-7 parts by mass of silica. This invention also provides a probe therefor having the same characteristics, including a case having at least one open end; the lens arranged so that a tip thereof is exposed at the end; a piezoelectric element housed in the case; an acoustic matching layer placed between the lens and the element; and a backing layer disposed opposite the acoustic matching layer with the element in between.

### TECHNICAL FIELD

The present invention relates to a lens for ultrasonic diagnosis apparatuses and a probe for ultrasonic diagnosis apparatuses. More specifically, the present invention relates to a lens for ultrasonic diagnosis apparatuses having requisite acoustic characteristics concerning sonic velocity, acoustic impedance and acoustic attenuation coefficient and exhibiting high abrasion resistance, and a probe for ultrasonic diagnosis apparatuses equipped with the lens.

### BACKGROUND ART

The ultrasonic diagnosis apparatus, such as electronic ultrasound scanners, is equipped with a probe in which a piezoelectric element, which is also called piezoelectric vibrator, is arranged. This probe is also provided with a lens for ultrasonic diagnosis apparatuses, capable of transmitting ultrasound into a subject and receiving it from the subject through contact with the subject. The lens for ultrasonic diagnosis apparatuses needs to have requisite acoustic characteristics concerning sonic velocity, acoustic impedance and acoustic attenuation coefficient. Thus the lens is usually made of silicone rubber which has acoustic impedance similar to the acoustic impedance of the subject and small acoustic attenuation coefficient for ultrasound, and through which acoustic waves propagate at sonic velocities of 1500 m/sec. or less. While the silicone rubber is excellent in the acoustic characteristics, it is inferior in some other properties such as chemical resistance and gas permeability. Lenses for ultrasonic diagnosis apparatuses, which are made of silicone rubber provided with a film on the surface thereof, have been proposed to improve those properties.

Silicone rubber lenses for ultrasonic diagnosis apparatuses, with a film on the surface thereof, are disclosed, for example, in Patent Documents 1-4. In more detail, Patent Document 1 teaches a film of polyimide resin or polyester resin for the film of the lenses; Patent Document 2 a protective film including fluororesin; Patent Document 3 a fluororubber layer; and Patent Document 4 a film made of a resin such as polyimide resin, fluororesin or polyester resin.

### PRIOR ART DOCUMENTS

### Patent Documents

Patent Document 1: JP S64-34559 U (1989)
Patent Document 2: JP H4-181896 A (1992)
Patent Document 3: JP 3038167 Y
Patent Document 4: JP H4-4497 U (1992)

### DISCLOSURE OF THE INVENTION

### Problems to be Solved by the Invention

The inventor of the present invention and his co-workers found that lenses with such a film as those disclosed in the patent documents did not work sufficiently as a lens for ultrasonic diagnosis apparatuses, because the lenses deteriorated in their acoustic characteristics and other favorable properties due to the presence of the film.

Also, the lens for ultrasonic diagnosis apparatuses is required to have high abrasive resistance in addition to the acoustic characteristics, because the lens is moved on the surface of the subject so that the apparatus is able to obtain images of the subject.

The objective of the present invention is to provide a lens for ultrasonic diagnosis apparatuses having requisite acoustic characteristics concerning sonic velocity, acoustic impedance and acoustic attenuation coefficient and exhibiting high abrasion resistance, and a probe for ultrasonic diagnosis apparatuses equipped with the lens.

### Means to Solve the Problems

The present invention, or means for solving the aforementioned problem, provides a lens for ultrasonic diagnosis apparatuses comprising a base formed from a silicone rubber composition, and a urethane coat layer placed on an outer surface of the base, wherein the urethane coat layer includes 100 parts by mass of a urethane resin and from 3 to 7 parts by mass of silica.

The lens for ultrasonic diagnosis apparatuses should further have the following features:

(1) The lens preferably has a mass change from 0.01 to 0.2% measured with the method that will be explained hereafter.
(2) The urethane coat layer is preferably made by applying a urethane resin composition including 100 parts by mass of the urethane resin and from 3 to 7 parts by mass of the silica, to the outer surface of the base and curing the applied urethane resin composition.
(3) The urethane coat layer preferably has a thickness from 10 to 50 μm.

### <Method of Measuring a Mass Change>

(1) Prepare a test piece in the shape of a disc with a diameter of 125 mm and a thickness of 2.015 mm, having a silicone rubber layer and a urethane coat layer placed on the silicone rubber layer, wherein the materials for the test piece are the same as those for the lens for ultrasonic diagnosis apparatuses. Mount the test piece on a fixing table of an abrasion tester according to JIS K6264-2, and fix it to the table.
(2) Fit two disc-shaped grindstones with a circumference of 160 mm and a thickness of 13 mm to the shafts of the abrasion tester, so that the common axis thereof is perpendicular to the planes including the axis of the test piece, and the distance between a first face of one grindstone and a second face of the other grindstone is 52 mm, wherein the first face is opposite the second face with the plane as center plane in between.
(3) Press the grindstones to the test piece so that the contact face between the grinding surface of each grindstone and the disc-shaped test piece is 1 mm by 13 mm in dimensions. Make the fixing table 3000 turns at a rotational speed of 60 r.p.m., with keeping the grindstones pressed to the test piece. Meanwhile the grindstones are rotated by the turns of the disc-shaped test piece.
(4) Measure the initial mass of the test piece before it is fixed to the fixing table and the final mass of the test piece after 3000 turns. Calculate the difference between the initial mass and the final mass. The mass change (%) is defined as the ratio of the difference to the initial mass in percentage terms.

The probe for ultrasonic diagnosis apparatuses, as means for solving the aforementioned problem, comprises a case having at least one open end; the lens for ultrasonic diagnosis apparatuses according to any one of claims 1-4, arranged so that a tip of the lens is exposed at the open end; a piezoelectric element housed in the case; an acoustic matching layer arranged between the lens and the piezoelectric element; and a backing layer disposed opposite the acoustic matching layer with the piezoelectric element in between.

### Advantages of the Invention

The lens for ultrasonic diagnosis apparatuses according to the present invention, comprising a base made of a silicone rubber composition, an outer surface of the base provided with a urethane coat layer including 100 parts by mass of a urethane resin and from 3 to 7 parts by mass of silica, is capable of exhibiting high abrasive resistance without substantial deterioration in the acoustic characteristics. Therefore the present invention provides a lens and a probe for ultrasonic diagnosis apparatuses showing high abrasive resistance as well as having requisite acoustic characteristics concerning sonic velocity, acoustic impedance and acoustic attenuation coefficient.

### BRIEF DESCRIPTION OF THE DRAWINGS

FIG. 1 shows schematic views of a first example of the lens for ultrasonic diagnosis apparatuses according to the present invention. FIG. 1(a) is a schematic front view of the first example, and FIG. 1(b) is a side view thereof.

FIG. 2 is a schematic sectional view showing a first example of the probe for ultrasonic diagnosis apparatuses to which the lens according to the present invention is fixed.

FIG. 3 shows schematic views of a second example of the lens for ultrasonic diagnosis apparatuses according to the present invention. FIG. 3(a) is a schematic front view of the second example, and FIG. 3(b) is a side view thereof.

FIG. 4 is a schematic sectional view showing a second example of the probe for ultrasonic diagnosis apparatuses to which the lens according to the present invention is fixed.

FIG. 5 is a schematic illustration explaining the method of measuring the mass change in a lens for ultrasonic diagnosis apparatuses.

### BEST MODE TO CARRY OUT THE INVENTION

The lens for ultrasonic diagnosis apparatuses according to the present invention comprises a base and a urethane coat layer. The lens is fixed to the tip of the probe of ultrasonic diagnosis apparatuses, which probe contacts the surface of a subject, e.g. a human, through a suitable medium such as olive oil or liquid paraffin. One example of the ultrasonic diagnosis apparatus may be an electronic ultrasound scanner.

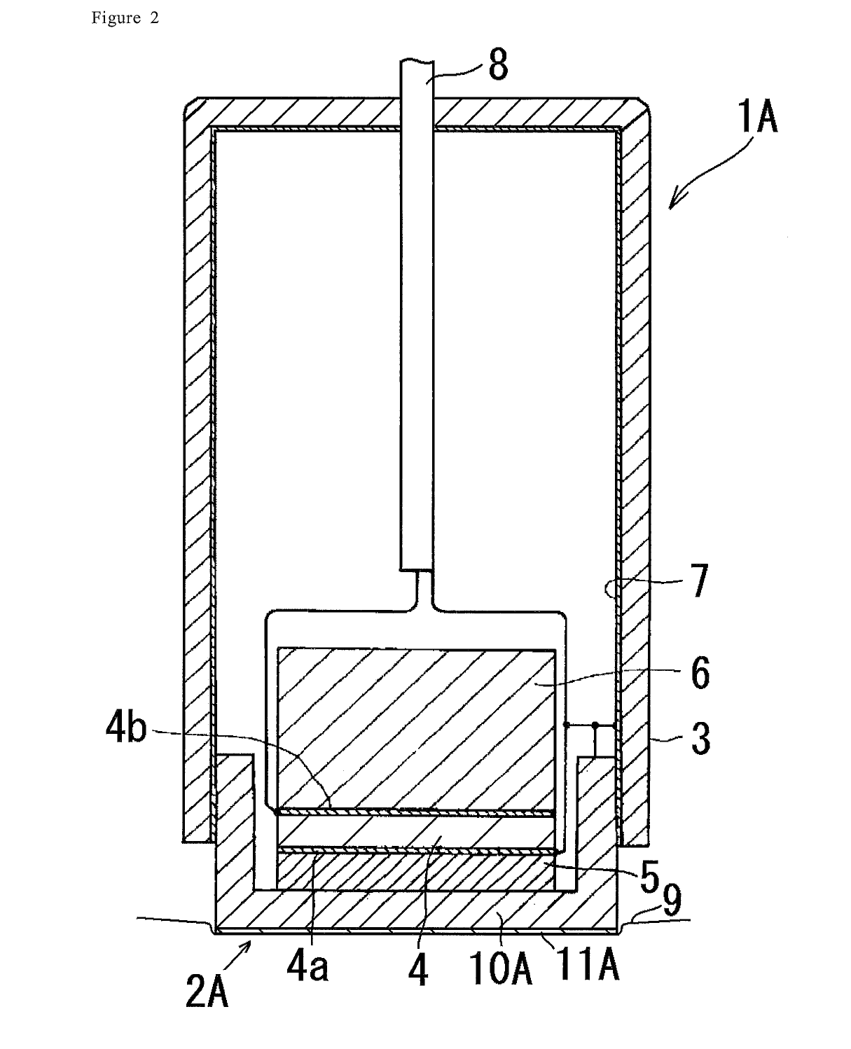

There is no limitation on the probe. One example, or a first example, of the probe may be a probe 1A comprising, as shown in FIG. 2, a case 3 in the shape of a general cylinder, at least one of whose ends is open; a lens 2A for ultrasonic diagnosis apparatuses, arranged so that a tip of the lens is exposed at the end of the case 3; a piezoelectric element 4 housed in the case 3; an acoustic matching layer 5 disposed between the lens 2A and the piezoelectric element 4; and a backing layer 6 opposite the acoustic matching layer 5 with the piezoelectric layer 4 in between. In more detail, the probe 1A has the case 3 in the shape of a general cylinder, at least one of whose ends is open; the lens 2A for ultrasonic diagnosis apparatuses arranged so that a tip of the lens 2A projects from the case 3 to the outside at the one end of the case; the piezoelectric element 4 housed in a space between the case 3 and the lens 2A; a first electrode 4a and a second electrode 4b on the respective surfaces of the piezoelectric element 4; the acoustic matching layer 5 formed on a surface of the first electrode 4a, which in turn is formed on the head-side surface of the piezoelectric element 4, the acoustic matching layer contacting the lens 2A; a backing layer 6, formed on a surface of the second electrode 4b, which in turn is formed on the rear-side surface of the piezoelectric element 4; a shielding film 7 with which the inner surface of the case 3 is provided; and leads 8 connecting the first electrode 4a and the second electrode 4b.

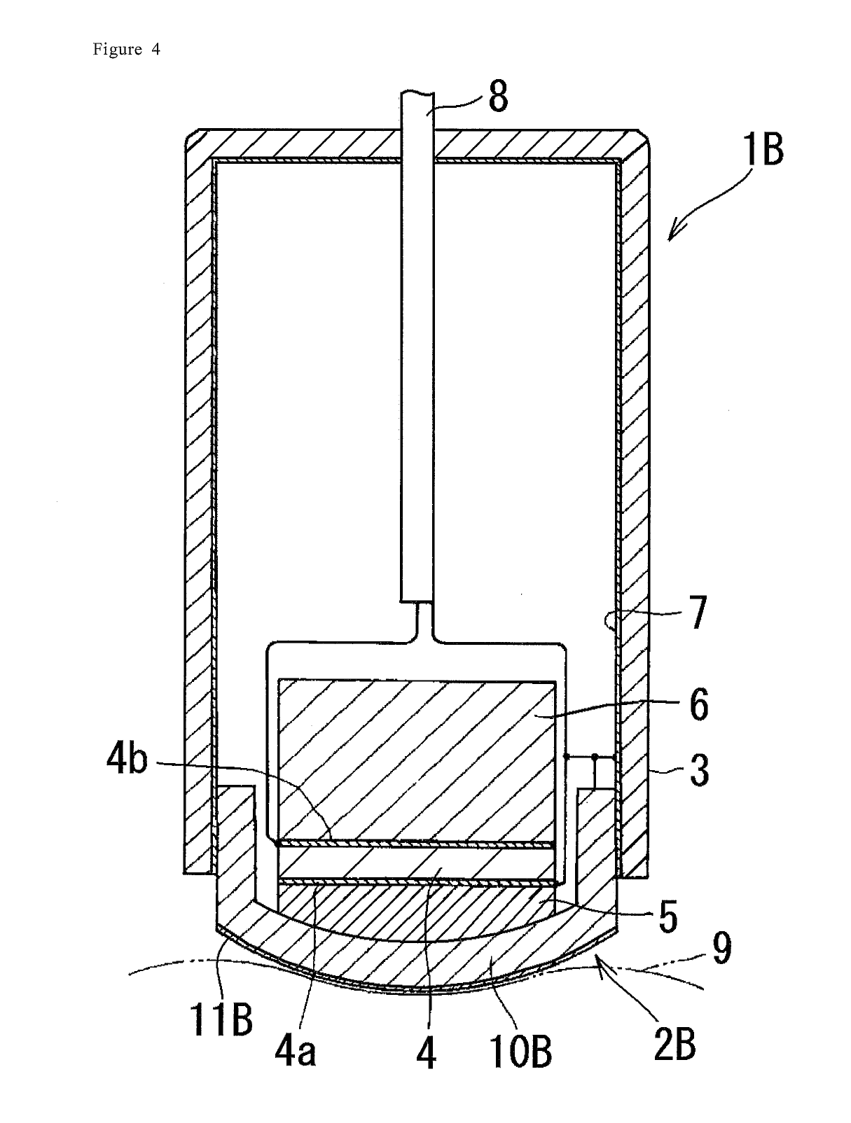

Another example, or a second example, of the probe may be a probe 1B comprising, as shown in FIG. 4, a case 3 in the shape of a general cylinder, at least one of whose ends is open; a lens 2B for ultrasonic diagnosis apparatuses, arranged so that a tip of the lens is exposed at the end of the case 3; a piezoelectric element 4 housed in the case 3; an acoustic matching layer 5 disposed between the lens 2B and the piezoelectric element 4; and a backing layer 6 opposite the acoustic matching layer 5 with the piezoelectric layer 4 in between. As understood, the probe 1B is essentially the same as the probe 1A, except that they are different from each other only in the shape of the lens for ultrasonic diagnosis apparatuses. Thus we will omit detailed explanations of the probe 1B; an element of the probe 1B having a referential numeral that is also used to refer to an element of the probe 1A is intended to be the same as the corresponding element of the latter.

For the probe such as those that have been explained hereinbefore may be employed known elements and materials without special limitations, except for the lenses 2A and 2B. Examples of the probe may be various probes for ultrasonic diagnosis apparatuses manufactured by GE Healthcare Company.

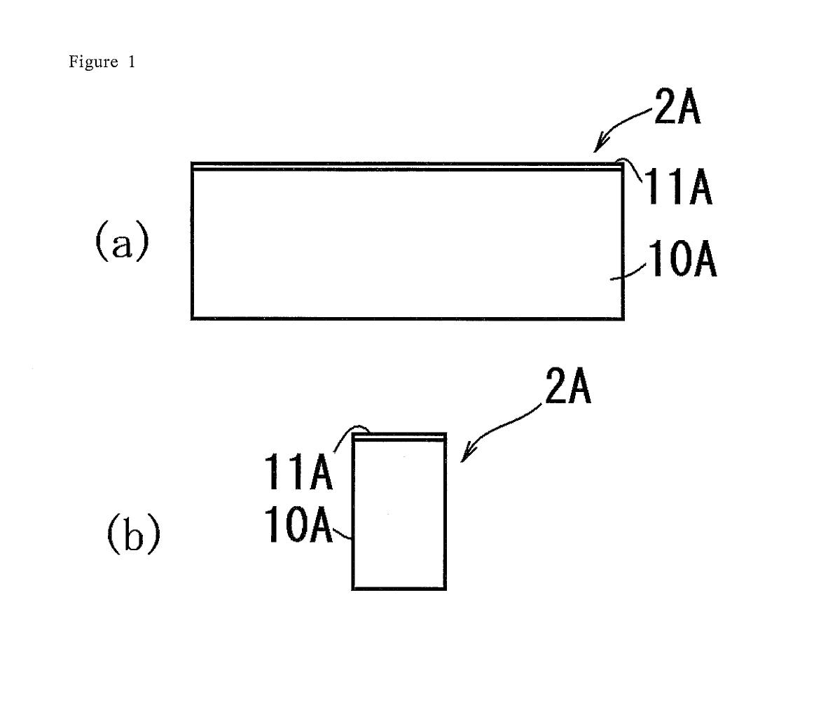

We will explain the lens 2A, which is an example of the lens for ultrasonic diagnosis apparatuses according to the present invention, referring to the attached figures. As shown in FIGS. 1 and 2, the lens 2A for ultrasonic diagnosis apparatuses comprises a base 10A and a urethane coat layer 11A applied onto an outer surface of the base 10A.

As shown in FIGS. 1 and 2, the lens 2A is in the shape of a rectangular-cuboid cylinder with one end open and the other closed. A portion with the open end of the cylinder is engaged with one end of the case 3. The shape and dimensions of the lens 2A may appropriately be adjusted according to the shape and dimensions of the case 3 to which the lens is fixed. There are no special limitations on the shape and dimensions of the lens. In this example, the lens 2A has a rectangular bottom with a flat surface, and four walls respectively standing vertically from the four edges. The thickness of each wall is the same as that of the bottom. The bottom has a planar outer face that is generally perpendicular to the axis of the bottom. The face makes the outer surface of the lens 2A on which the urethane coat layer 11A is formed and placed, and the urethane coat layer 11A contacts the surface of a subject 9. The urethane coat layer will be explained hereafter.

As shown in FIGS. 1 and 2, the base 10A, the main part of the lens 2A, is essentially the same as the lens 2A, except that the urethane coat layer 11A is not formed on it. In other words, the base 2 is the lens base for a lens of ultrasonic diagnosis apparatuses. The base is a tube with one end closed, in the shape of a rectangular cuboid. Specifically, as shown in FIGS. 1 and 2, the base 2 has a bottom in the shape of a rectangle with a flat surface and four side walls projecting perpendicularly from the respective edges of the bottom. The thickness of each side wall is the same as that of the bottom. The base 2 is fitted to the opening of the case 4, which makes the base also called cap.

The dimensions of the base 10A are suitably adjusted to those of the case 3 to which it is fitted, and there is no special limitation on them. However, because the dimensions affect the transmittance and receipt of ultrasonic waves, the bottom of the base 10A should not have an excessively large thickness. For example, the thickness should be adjusted to 0.1 to 2.4 mm. This base is made of a silicone rubber composition, which will be explained hereinafter.

The urethane coat layer 11A is a thin film including 100 parts by mass of a urethane resin and from 3 to 7 parts by mass of silica. The layer is placed on the outer surface of the base 10A. There is no special limitation on the way of forming the urethane coat layer, as long as at least the outer surface of the base 10A, which contacts the surface of a subject 9, is covered with the layer, as understood from FIGS. 1 and 2. In addition to the outer surface of the bottom, the outer surface of the portion of the side walls that projects out of the case 3 may be provided with the urethane coat layer 11A. Alternatively, the entire outer surface of the base 10A may be provided with the urethane coat layer 11A. The whole outer surface of the bottom of the base 10A is provided with the urethane coat layer 11A. In other words, the whole outer surface of the bottom thereof is covered with the urethane coat layer 11A.

The urethane coat layer 11A is made by curing a urethane resin composition, which will be explained hereinafter. The urethane coat layer 11A is formed, for example, by applying the urethane resin composition, which will be explained hereinafter, onto the outer surface of the base 10A, and then heating and curing the applied urethane resin composition. Therefore the urethane coat layer 11A includes a urethane resin formed from the urethane resin composition. This urethane resin is made from a polyol and a polyisocyanate, the combination of which is capable of forming a urethane resin. There is no special limitation on the kinds of the polyol and the polyisocyanate. The urethane coat layer 11A may include, other than the urethane resin, various additives, which are normally added to various urethane resin compositions.

For the silica may be employed silicas such as fumed silica or precipitated silica. Surface-treated silica may also be employed. The inclusion of the silica in the urethane coat layer 11A improves the abrasion resistance of the lens 2A for ultrasonic diagnosis apparatuses. It may make the urethane resin, which generally has low adhesion to the base 10A, tightly adhere to the base 10A depending on occasions. The silica may be used in a suitable form such as droplets, granules, or powder, the mean diameter of which should be not more than the thickness of the urethane coat layer 11A, preferably from 5 to 10 μm for example. The mean diameter of silica may be measured by a sieve method.

The amount of the silica included in the urethane coat layer 11A is from 3 to 7 parts by mass to 100 parts by mass of the urethane resin. When the amount is less than 3 parts by mass, the urethane coat layer may not sufficiently show the advantages due to the inclusion of the silica and it may be inferior in abrasion resistance. The shortage may sometimes cause deterioration in adhesion between the base 10A and the urethane coat layer 11A. On the other hand, when the amount exceeds 7 parts by mass, the acoustic characteristics may sometimes be lowered. From the viewpoint of satisfactory achievement of the objectives of the present invention, the amount of the silica should preferably be from 4 to 8 parts by mass. The amount of the silica in the urethane coat layer 11A may be measured by the thermogravimetry according to JIS K7120. Specifically, the urethane coat layer 11A is heated to 900° C. Then, the mass of the residue is measured. The portion of the urethane coat layer that has been burned up by the heating is considered to be the urethane resin. The amount of the silica is defined as the proportion of the mass of the residue to the mass of the burned-up portion, wherein the mass of the burned-up portion, or the urethane resin, is regarded as 100 parts by mass.

The urethane coat layer 11A should preferably have a thickness from 10 to 50 because the thickness in this range enables the base 10A made of the silicone rubber composition to maintain its excellent acoustic characteristics. It is particularly preferable when the layer has a thickness from 10 to 30 μm.

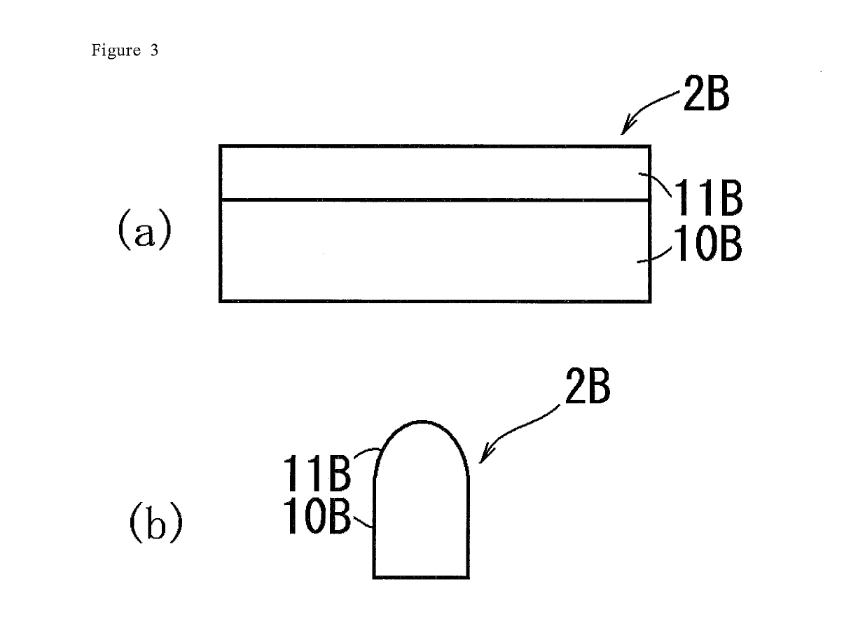

Referring to the figures, we will explain a lens 2B, which is another example of the lens for ultrasonic diagnosis apparatuses according to the present invention. As shown in FIGS. 3 and 4, the lens 2B comprises a base 10B and a urethane coat layer 11B applied onto an outer surface of the base 10B.

The lens 2B is essentially the same as the lens 2A, except that they are different in shapes, and may also be different in sizes depending on the situation. Specifically, as shown in FIGS. 3 and 4, the lens 2B is in the shape of a rectangular-cuboid tube, a section of which, taken along a plane perpendicular to the axis thereof, is in the shape of a general rectangle, and both ends of the tube are open. The lens also has a tip projecting from one end along the axis in the opposite direction of the other end. The thickness of this tip is the same as that of the side walls, which form the tube, as shown in FIG. 4. The outer face of the tip is in the shape of a semi-cylinder whose axis runs parallel with the longitudinal sides of the rectangular section. In other words, the tip becomes gradually thinner along the axis of the tube, from the end of the tube to the farthest edge of the tip, while it lies unchanged along the longitudinal sides of the rectangular section. This semi-cylindrical surface makes the outer surface of the lens 2B for ultrasonic diagnosis apparatuses on which the urethane coat layer 11B is placed and formed. The urethane coat layer 11B formed on the semi-cylindrical surface contacts the surface of a subject 9.

As shown in FIGS. 3 and 4, the base 10B, the body of the lens 2B, has essentially the same shape as the lens 2B for ultrasonic diagnosis apparatuses. There is no special limitation on the method of forming the urethane coat layer, as long as at least the outer surface of the base 10B, which contacts the surface 9 of a subject, is covered with the layer. In addition to the semi-cylindrical surface, the outer surface of the portion of the side walls that projects out of the case 3 may be provided with the urethane coat layer 11B. Alternatively, the entire outer surface of the base 10B may be provided with the urethane coat layer 11B. The whole outer surface of the semi-cylindrical surface of the base 10B is provided with the urethane coat layer 11B. In other words, the whole semi-cylindrical surface thereof is covered with the urethane coat layer 11B.

The sonic velocity c in the lenses 2A and 2B for ultrasonic diagnosis apparatuses with the configurations, which may sometimes be called “lens 2 for ultrasonic diagnosis apparatuses”, should preferably be from 900 to 1600 m/s, which is close to the sonic velocity in biological tissue. The sonic velocity in the lens 2A for ultrasonic diagnosis apparatuses is measured in the following way: A plate-shaped test piece is cut out of the lens 2 or is prepared with the same method as in the preparation of the lens 2. An ultrasonic wave, a longitudinal wave whose frequency is 5 MHz, is emitted into the test piece from the surface covered with the urethane coat layer 11A or 11B, which may sometimes be called urethane coat layer 11. The wave is reflected by the other surface, or more precisely the back of the other surface, and returned to the covered surface. The time period from the emission to the return is measured with an ultrasonic thicknessmeter named “Echometer 1060”, manufactured by NIHON MATECH CORPORATION. The measured time period is divided by the thickness t of the test piece.

The lens 2 for ultrasonic diagnosis apparatuses should have an acoustic impedance from 1.30 to 1.60 (kg/(m²·S×10⁶)), which is close to the acoustic impedance of a living organism, because the impedance within this range minimizes the reflection of ultrasonic waves at the interface between a living organism and the acoustic lens. The acoustic impedance may be calculated from the specific gravity ρ (g/cm³) of the lens 2 for ultrasonic diagnosis apparatuses and the sonic velocity c (m/s), according to the equation: ρ×c. The division of the mass (g) of the lens 2 by the volume (cm³) thereof provides the specific gravity ρ.

The lens 2 for ultrasonic diagnosis apparatuses should have a small acoustic attenuation coefficient for a longitudinal wave at a frequency from 1 to 5 MHz. For example, the lens should preferably have an acoustic attenuation coefficient of 3.0 dB/mm (at 4.5 MHz) or less for a 4.5 MHz longitudinal wave, more preferably 2.6 dB/mm or less, particularly preferably 1.1 to 1.7 dB/mm. The acoustic attenuation coefficient may be calculated according to the equation: 20×log₁₀ [(Intensity B1/Intensity B2)/(Thickness×2)]. In this equation, Intensity B1 is defined as the intensity of a supersonic wave with a frequency of 4.5 MHz when it is received by one surface of the lens 2, which wave has been emitted into the lens for ultrasonic diagnosis apparatuses from the one surface toward the other surface so that it would perpendicularly traverse the lens 2, reflected by the back of the other surface, and returned to the one surface. In other words, Intensity B1 is the intensity when a supersonic wave, having been emitted into the lens 2 from one surface thereof, returns to the emitted point after a round trip along the thickness thereof. Intensity B2 is the intensity when a supersonic wave, having been emitted into the lens 2 from one surface thereof, returns to the emitted point after two round trips along the thickness thereof. The thickness is the thickness of the lens at the point at which the ultrasonic wave is emitted. More specifically, the thickness is the total of the thickness of the urethane coat layer and that of the base 10A at the position where the ultrasonic wave is emitted. When the lens 2A is employed, the thickness of the base is that of the bottom. When the lens 2B is employed, the thickness of the base is that of the tip, more specifically that of the center of the tip. The acoustic attenuation coefficient may be measured with, for example, an ultrasonic thicknessmeter named “Echometer 1060”, manufactured by NIHON MATECH CORPORATION.

The lens 2 for ultrasonic diagnosis apparatuses has excellent abrasion resistance. Specifically, the lens 2 should preferably have a mass change from 0.01 to 0.2%, particularly preferably from 0.01 to 0.1%, measured with the test that will be explained in the following. The lens with the mass change within this range is capable of maintaining the initial state of the urethane coat layer 11, as well as the base 10A or 10B, which may sometimes be called base 10, for a long time, even when it is fixed to a probe for ultrasonic diagnosis apparatuses and moved on the surface of subjects 9.

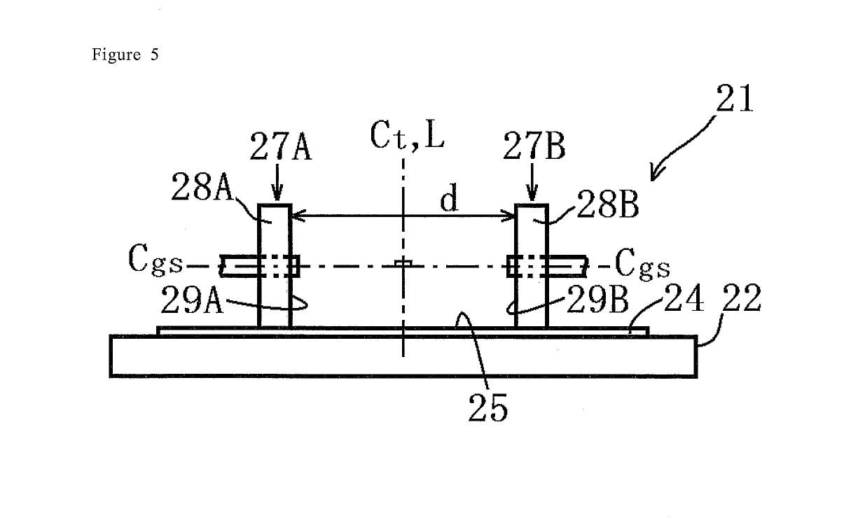

The mass change may be obtained with an abrasion tester according to JIS K6264-2, such as a Taber abrasion tester manufactured by Yasuda Seiki seisakusho, LTD. by the following method. FIG. 5 is a schematic illustration that shows the method of measuring the mass change.

First, prepare the same materials as the materials of the lens 2 for ultrasonic diagnosis apparatuses whose mass change will be measured. Specifically, identify the constituents and their respective amounts of the base 10 of the lens 2 and those of the urethane coat layer 11 thereof by suitable methods such as chemical analysis. Prepare a material that has the same constituents and their respective amounts as the base has and a material that has the same constituents and their respective amounts as the urethane coat layer has. Cure each of the materials under suitable conditions, and make a disc-shaped test piece 24, which is 125 mm in diameter and 2.015 mm in thickness, comprising a silicone rubber layer and a urethane coat layer placed on the silicone rubber layer. The respective thicknesses of the silicone rubber layer and the urethane coat layer of this disc-shaped test piece 24 may be the same as or different from those of the silicone rubber layer and the urethane coat layer of the lens 2 whose mass change will be measured, as long as the mass change is capable of being measured. For example, the thickness of the silicone rubber layer of the disc-shaped test piece 24 may be set to 2.000 mm and that of the urethane coat layer 11 thereof to 0.015 mm. Measure the mass of the disc-shaped test piece 24 as prepared, which may be called initial mass hereinafter. Mount the disc-shaped test piece 24 on a fixing table 22 of an abrasion tester 21 and fix it to the table. The surface 25 of the disc-shaped test piece 24 has the same surface roughness Rz as the lens 2 for ultrasonic diagnosis apparatuses has.

Prepare two disc-shaped grindstones 27A and 27B. Each of the grindstones is in the shape of a disc with a cylindrical face, having a circumference of 160 mm and a thickness of 13 mm. The cylindrical faces serve as grinding surfaces 28A and 28B. An example of the disc-shaped grindstones 27A and 27B may be a grindstone of model CS-17, manufactured by Taber Industries. Fix the two disc-shaped grindstones 27A and 27B onto the shafts of the abrasion tester 21, so that the common axis C_{gs} of the grindstones are perpendicular to a plane L that includes the axis Cₜ of the disc-shaped test piece 24 on the fixing table 22, or the axis C_{gs} is parallel with the surface 25 of the disc-shaped test piece 24, as shown in FIG. 5. Furthermore, arrange the two disc-shaped grindstones 27A and 27B on the shafts of the abrasion tester 21, so that the respective faces 29A and 29B of the grindstones 27A and 27B, with the face 29A opposite the face 29B, are disposed apart a distance d of 52 mm, with the plane L, as the central plane, in between.

Press the disc-shaped grindstones 27A and 27B onto the surface 25 of the disc-shaped test piece 24, so that the contact faces between the grinding surfaces 28A and 28B of the grindstones and the surface 25 have a width of 13 mm along the thickness of the grindstones and a length of 1 mm along the circumference thereof. Make the fixing table 22 with the disc-shaped test piece 24 thereon 3000 turns at a rotational speed of 60 r.p.m. in one direction, with keeping the grindstones pressed to the test piece. Meanwhile the disc-shaped grindstones 27A and 27B are rotated on the surface 25 of the disc-shaped test piece 24, with the axis C_{gs} as a pivot, by the turns of the test piece 24.

After the 3000 turns of the fixing table 22, remove the disc-shaped test piece 24 from the fixing table 22, and measure the mass of the test piece, which may sometimes be calls mass after 3000 turns. Subtract the mass after 3000 turns from the initial mass. The percentage of the remainder to the initial mass is regarded as the mass change (%) of the lens 2 for ultrasonic diagnosis apparatuses.

In place of, or in addition to the mass change, the lens 2 for ultrasonic diagnosis apparatuses should preferably have a thickness change from 0.1 to 13%, particularly preferably from 0.1 to 11%, at parts thereof including a portion contacting the surface of a subject 9. The lens with the thickness change within this range is capable of maintaining the initial state of the urethane coat layer 11, as well as the base 10, for a long time, even when it is fixed to a probe for ultrasonic diagnosis apparatuses and moved on the surface of subjects 9. The thickness change may be obtained with the same procedure as in obtaining the mass change, except that the initial thickness of the urethane coat layer and the thickness after 3000 turns thereof are measured. The thickness after 3000 turns is measured at the portion of the test piece to which the disc-shaped grindstones 27A and 27B are pressed. The thickness of the urethane coat layer may be measured with a film thickness non-contact measurement system such as a VK-8700 Generation II 3D Laser Scanning microscope, manufactured by Keyence Corporation.

The portion of lens 2 that contacts the surface of a subject 9 should preferably have a thickness from 0.1 to 2.5 mm in order to minimize the attenuation of the ultrasonic energy emitted by the oscillator.

The outer surface of the lens 2, more specifically that of the urethane coat layer 11, particularly that of the portion contacting the surface of a subject 9 should have a surface roughness Rz preferably from 6 to 15 μm, particularly preferably from 8 to 12 μm. The surface roughness Rz is represented by a ten-point height of irregularities according to JIS B0601-1994. The roughness may be measured by the JIS B0601-1994 method with a Gaussian filter under the conditions where the tip diameter φ of the stylus for rubber is 5 μm, the load is 0.07 g, the velocity is 0.12 mm/sec, the measured length 0.4 mm, the thickness of the test piece is 2.015 mm, and the cutoff wavelength is 0.8 mm.

In the following, we will explain the method of producing the lens for ultrasonic diagnosis apparatuses according to the present invention. The method for producing the lens may suitably be selected from the following ones depending on the situation:

Prepare a base 10 with a silicone rubber, apply a urethane resin composition to the outer surface of the base 10, and cure the composition

Prepare a thin film of a urethane resin composition and stick it to the outer surface of a base 10 made of a silicone rubber composition

Integrally mold the thin film and the silicone rubber composition.

Among these methods preferable is the method which comprises preparing a base 10 with a silicone rubber, applying a urethane resin composition to the outer surface of the base 10, and curing the composition. A primer may be applied to the outer surface of the base 10.

There is no special limitation on the silicone rubber composition, as long as it includes silicone rubber. A typical example of the silicone rubber composition may be those including silicone rubbers with a dimethylpolysiloxane structure including vinyl groups, silica and vulcanizing agents. When used in the method of preparing a lens for ultrasonic diagnosis apparatuses according to the present invention, which will be explained hereinafter, the silicone rubber composition should be so-called ‘milable’ ones. For the vulcanizing agent may be employed those generally used as a vulcanizing agent for silicone rubber, without any special limitations. A specific example of the silicone rubber composition for the base 10 may be “a silicone rubber composition comprising a silicone rubber with a dimethylpolysiloxane structure including vinyl groups; silica particles with a weight-average mean diameter from 15 to 30 nm in an amount from 40% by mass to 50% by mass, to the mass of the silicone rubber; and 2,5-dimethyl-2,5-di(tert-butylperoxy)hexane as a vulcanizing agent” disclosed in the JP 3,468,753 patent publication.

More specific examples of the silicone rubber composition including the silicone rubber and silica (SiO₂) may be the commercial products whose names are “KE-981U”, “KE-971U”, “KE-752U” and “KE-772”, all of which are manufactured by Shin-Etsu Chemical Co., Ltd.

The urethane resin composition includes 100 parts by mass of a urethane resin or a precursor capable of producing a urethane resin, and from 3 to 7 parts by mass of the silica to 100 parts by mass of the urethane resin or precursor. The precursor may include the combination of a polyol and a polyisocyanate, or a urethane prepolymer made through the reaction of these compounds. The silica included in the urethane resin composition was explained hereinbefore.

The polyol includes various polyols having at least two hydroxyl groups in a molecule, preferably at the ends thereof, which are usually used for the production of polyurethane. The polyol should preferably be at least one selected from polyether polyols and polyester polyols, with polyester polyols more preferable because they are excellent in thermal stability. Examples of the polyether polyols may include polyalkylene glycols such as polyethylene glycol, polypropylene glycol and polypropylene glycol-ethylene glycol; polytetramethylene ether glycol; copolymer polyols of tetrahydrofuran and an alkyleneoxide; and various modified compounds and mixtures thereof. Examples of the polyester polyols may include polyester polyols provided by condensation of a dicarboxylic acid, such as adipic acid, and a polyol, such as ethylene glycol and hexanediol; lactone polyester polyols; polycarbonate polyols; and mixtures thereof.

The polyols should preferably be diols. Therefore they should more preferably be polyester diols or polyether diols, with polyester diols particularly preferable. The polyol should have preferably a number average molecular weight from 800 to 15000, more preferably from 1000 to 5000. The number average molecular weight is a molecular weight by gel permeation chromatography (GPC), converted to polystyrene standard. The polyols may be used singly or in combination. Also, combinations of a polyether polyol and a polyester polyol may be employed.

Various polyisocyanates having at least two isocyanate groups in a molecule, preferably at the ends thereof, which are usually used for the production of polyurethane would suffice for the isocyanate. It may include, for example, aliphatic polyisocyanates, aryl polyisocyanates and derivatives thereof. Examples of the aryl polyisocyanate may include xylylene diisocyanate (XDI), diphenylmethane diisocyanate (MDI), toluene diisocyanate, which is also called tolylene diisocyanate (TDI), 3,3′-bitolylene-4,4′-diisocyanate, 3,3′-dimethyldiphenyl-methane-4,4′-diisocyanate, 2,4-tolylene diisocyanate uretidinedione (a dimer of 2,4-TDI), xylene diisocyanate, naphthalene diisocyanate (NDI), p-phenylene diisocyanate (PDI), tolidine diisocyanate (TODI), and m-phenylene diisocyanate. Examples of the aliphatic polyisocyanate may include hexamethylene diisocyanate (HDI), 4,4′-dicyclohexylmethane diisocyanate (hydrogenated MDI), o-toluidine diisocyanate, lysine diisocyanate methyl ester, isophoronediisocyanate (IPDI), norbornane diisocyanate methyl, trans-cyclohexane-1,4-diisocyanate, and triphenylmethane-4,4′,4″-triisocyanate. The derivatives may include multimers of the polyisocyanate, polyisocyanates reacted with e.g. a small amount of a polyol, including urethane prepolymers, dimers resulting from the formation of uretidione, isocyanurates, carbodimides, uretonimine, allophanate, urea, and biuret.

The polyisocyanates should preferably be diisocyanates, with aliphatic polyisocyanates particularly preferable. The polyisocyanate should have a molecular weight of preferably from 500 to 2000, more preferably from 700 to 1500. The polyisocyanates may be used singly or in combination.

The urethane resin and the urethane prepolymer may be obtained through the reaction of the polyol and the polyisocyanate that are mixed in a mixing proportion to be explained hereinafter. The mixing proportion should preferably be such that the molar ratio (NCO/OH) of isocyanate groups (NCO) included in the polyisocyanate to hydroxyl groups (OH) included in the polyol is from 0.7 to 1.15. This molar ratio (NCO/OH) should preferably be from 0.85 to 1.10, because the molar ratio within this range leads to the prevention of hydrolysis of polyurethane. In actual production, however, the amount of polyisocyanate may be from three to four times as large as the amount that falls within the proper molar ratio, in view of working environment and errors during the operation.

The urethane resin composition may include, in addition to the urethane resin and/or the precursor capable of producing a urethane resin and silica, solvents, and auxiliaries that are usually used in the reaction between a polyol and a polyisocyanate, such as chain extenders and crosslinking agents, if desired. Examples of the chain extenders and crosslinking agents may include glycols, hexanetriol, trimethylolpropane and amines.

The urethane resin composition may be obtainable by mixing 100 parts by mass of the urethane resin or precursor capable of producing a urethane resin, from 3 to 7 parts by mass of silica, and a solvent and auxiliaries each in a suitable amount, by an appropriate method.

Preferable methods for producing the lens for ultrasonic diagnosis apparatuses will be explained in detail. Methods of using molds are normally selected for forming a base 10 from the silicone rubber composition. For example, a compression molding of the silicone rubber composition, which is placed between a first mold with a projection and a second mold with a recess into which the projection fits, produces a base 10. The shape of the gap between the projection and the recess is the same as that of the base 10.

At the same time as, or subsequent to the compression molding of the silicone rubber composition, the composition is heated under the conditions where silicone rubber compositions are cured. For example, the silicone rubber compositions that have been explained hereinbefore become cured by heating at a temperature of 155° C. or more for about 3 to 10 minutes. A secondary heating may be carried out, if desired, after the silicone rubber composition is cured in this way.

The compression-molded is taken out of the molds after the composition is cured, and deburred if desired. The resultant is a base 10.

Onto the outer surface of the base 10 thus produced is applied the urethane resin composition including 100 parts by mass of the urethane resin and from 3 to 7 parts by mass of silica. The application of the urethane resin composition may be carried out by known methods, such as spraying, brush coating or dipping. Then, the urethane resin composition on the outer surface of the base 10 is cured. The urethane resin composition may be cured by heat or moisture.

The lens 2 for ultrasonic diagnosis apparatuses may be produced in this way.

The lens for ultrasonic diagnosis apparatuses, with the aforementioned features, shows high abrasion resistance without largely deteriorating in the acoustic characteristics. Therefore this invention is capable of providing lenses for ultrasonic diagnosis apparatuses and probes therefor which have high abrasion resistance as well as the requisite acoustic characteristics concerning sonic velocity, acoustic impedance and the acoustic attenuation coefficient.

Also, because the base 10 of these lenses 2 is made of the silicone rubber with the thin urethane coat layer 11 on the outer surface thereof, it has excellent tribological property on the surface of a subject 9, or human skin, and its contact with the surface of a subject 9 is satisfactory. Furthermore, the lens 2 has good adhesion to the base 10, because the urethane coat layer 11 includes silica. The lens follows changes of the base 10, such as deformations thereof, very well during its use, which leads to excellent durability.

The lenses for ultrasonic diagnosis apparatuses of the present invention are not limited to the foregoing examples, but are able to be variously modified within the gist and spirit of the present invention, or as long as the objective of the present invention is capable of being achieved.

For example, it is not mandatory for the lens to be in the shape of a quadrangular prism, but may be in the shape of a column, an elliptic cylinder or a polygonal prism, with one end thereof open. Alternatively, the lens may be formed from a single plate, or may be in the shape of a bent or folded plate, such as a V-shaped or U-shaped one with no opposite side faces.

In the foregoing examples, the base 10 and the urethane coat layer 11 of the lens 2 comprise single layers. However, the base and the urethane coat layer of the present invention may comprise multiple layers.

In the foregoing examples, the urethane coat layer 11 is directly placed on the outer surface of the base 10. However, the base 10 of the lens 2 for ultrasonic diagnosis apparatuses may be provided with the urethane coat layer 11 on the outer surface thereof with an adhesive layer or a primer layer in between.

### EXAMPLES

### Working Example 1

For the silicone resin composition was prepared a silicone rubber composition comprising 100 parts by mass of a silicone rubber preparatory composition that includes a silicone rubber and silica, a product named “KE-772” manufactured by Shin-Etsu Chemical Co., Ltd., and 0.5 part by mass of a vulcanizing agent, a product named “C-8” manufactured by Shin-Etsu Chemical Co., Ltd. This silicone rubber composition had a plasticity number of 420, measured according to JIS K6249.

For the urethane resin composition was prepared a urethane resin coating composition having the following composition:

A urethane resin preparatory composition including 100 parts by mass of polyester polyol and 12 parts by mass of isocyanurate-polyisocyanate, where the ratio [NCO/OH] of [NCO] to [OH] was 1.1/1 . . . 100 parts by mass

Silica: Fumed silica (Mean particle diameter: 3 μm). 5 parts by mass

Solvent: Paint thinner . . . 40 parts by mass

Then, a first mold having a projection whose section was in the shape of a general rectangle with the dimensions 15×23 mm and whose height was 3.0 mm, and a second mold having a recess whose section was in the shape of a general rectangle with the dimensions 16×24 mm and whose depth was 3.4 mm were prepared.

The silicone rubber composition was placed on the projection of the first mold, and the second mold was disposed so that the first mold would be overlaid with the second one. The second mold was moved toward the first mold to compress the silicone resin composition. At the same time, the engaged molds with the silicone resin composition inside were heated at 180° C. for 5 minutes, and thus the silicone resin composition was heated and cured. After the heat was dissipated, the molded article was taken from the molds. A base 10A with a thickness of 2.000 mm was produced.

The urethane resin coating composition was sprayed onto the base 10A, so that the thickness of the film after cure would be 15 μm. Then, the composition was cured in an environment of 150° C. and 5% relative humidity. A lens 2A for ultrasonic diagnosis apparatuses of Working Example 1 was thus produced. This lens 2A was 2.015 mm in total thickness of the base 10A and the urethane coat layer 11A at the portion to contact the surface of a subject 9.

### Working Example 2

A lens 2A for ultrasonic diagnosis apparatuses of Working Example 2 was produced by essentially the same method as in Working Example 1, except that the urethane resin coating composition was applied to the base, so that the thickness of the film after cure would be 30 μm.

### Comparative Example 1

A lens for ultrasonic diagnosis apparatuses of Comparative Example 1 was produced by essentially the same method as in Working Example 1, except that a fluororesin PTFE coating composition (product name: FLUON PTFE, manufactured by Asahi Glass Co., Ltd.), wherein “PTFE” stands for polytetrafluoro-ethylene, was used in place of the urethane resin coating composition.

### Comparative Example 2

A lens for ultrasonic diagnosis apparatuses of Comparative Example 2 was produced by essentially the same method as in Working Example 1, except that a polyimide resin coating composition (product name: PW-1000, manufactured by Toray Industries, Inc.) was used in place of the urethane resin coating composition.

### (Measurement of Sonic Velocity, Specific Gravity ρ, Acoustic Attenuation Coefficient and Surface Roughness Rz, and Calculation of Acoustic Impedance)

A plate-shaped test piece with a thickness t of 2.000 mm was produced by the same method as the lenses for ultrasonic diagnosis apparatuses were produced in Working Examples 1 and 2 and Comparative Examples 1 and 2. The sonic velocity c (m/s) and the specific gravity ρ were measured with this test piece by the methods explained hereinbefore. The acoustic impedance (kg/(m²·S×10⁶)) of this test piece was calculated. The acoustic attenuation coefficient (dB/mm) and the surface roughness Rz were measured with the produced lenses by the methods explained hereinbefore. The results are shown in Table 1.

### (Silica Content)

The silica content of the urethane coat layer of each produced lens was measured by the method explained hereinbefore. As a result, the silica content was almost the same as that of the urethane resin composition. The measured values are shown in Table 1.

### (Abrasion Resistant Test: Measurement of Mass Change)

A disc-shaped test piece 24 was made by using the silicone rubber composition that was formed into the base in each working or comparative example, and the material that was formed into the coat layer in each example. The test piece 24 had a 2.000-mm-thick silicone rubber layer and a 0.015-mm-thick coat layer on the silicone rubber layer. The conditions for curing the test pieces were the same as those for curing the lenses produced in the examples. Each disc-shaped test piece 24 had a diameter of 125 mm and a thickness of 2.015 mm. The mass change of these test pieces was measured by the method explained hereinbefore. The disc-shaped grindstones 27A and 27B were pressed onto the test piece 24. The applied pressure was about 1 kN. The contact face between each of the grinding surfaces 28A, 28B and the surface 25 of the test piece 24 was adjusted to have the dimensions 1 mm×13 mm.

### (Abrasion Resistant Test: Measurement of Thickness Change)

The initial thickness, prior to the measurement of the mass change above, of the coat layer of each disc-shaped test piece 24 and the thickness after 3000 turns thereof, subsequent to the measurement of the mass change, were measured at the portion of the test piece to which the disc-shaped grindstones 27A and 27B were pressed, by the method explained hereinbefore. The thickness change of each test piece 24 was calculated. The obtained was regarded as the thickness change of each lens for ultrasonic diagnosis apparatuses. The results are shown in Table 1.

TABLE 1

| | W. | W. | Com. | Com. |
|---|---|---|---|---|
| | Ex. 1 | Ex. 2 | Ex. 1 | Ex. 2 |
| Sonic velocity c (m/s) | 1000 | 979 | 853 | 924 |
| Specific gravity ρ (g/cm³) | 1.39 | 1.39 | 1.40 | 1.40 |
| Acoustic impedance | 1.40 | 1.36 | 1.20 | 1.30 |
| (kg/(m² · S × 10⁶) | | | | |
| Acoustic attenuation coefficient | 2.6 | 2.6 | Very | 2.7 |
| (dB/mm) | | | large | |
| Surface roughness Rz (μm) | 11.0 | 12.0 | 5.0 | 4.0 |
| Silica content (parts by mass) | 5.2 | 5.2 | 0 | 0 |
| Mass change (%) | 0.05 | 0.2 | 1.0 | 1.5 |
| Thickness change (%) | 4.2 | 11 | 15 | 20 |

As shown in Table 1, the lenses for ultrasonic diagnosis apparatuses produced in Working Examples 1 and 2 sufficiently satisfied the requisite acoustic characteristics concerning sonic velocity, acoustic impedance and the acoustic attenuation coefficient. They also endured the abrasion resistant tests under the severer conditions than actual conditions under which the lenses were normally used, and showed the small mass changes and thickness changes. It was obviously surmised that they would exhibit sufficient abrasion resistance in actual usage.

### INDUSTRIAL APPLICABILITY

The lens for ultrasonic diagnosis apparatuses and the probe therefore according to the present invention will favorably be used in ultrasonic diagnosis apparatuses, such as electronic ultrasound scanners.

### EXPLANATION OF REFERENCE NUMERALS

1A, 1B probe
2A, 2B lens for ultrasonic diagnosis apparatuses
3 case
4 piezoelectric element
4a, 4b electrodes
5 acoustic matching layer
6 backing layer
7 shielding film
8 lead
9 surface of a subject
10A, 10B base (cap)
11A, 11B urethane coat layer
21 abrasion tester
22 fixing table
24 disc-shaped test piece
25 surface
Cₜ axis
L plane
27A, 27B disc-shaped grindstone
28A, 28B grinding surface
29A, 29B face
C_{gs} common axis
d distance

1. A lens for ultrasonic diagnosis apparatuses comprising a base formed from a silicone rubber composition, and a urethane coat layer placed on an outer surface of the base, wherein the urethane coat layer includes 100 parts by mass of a urethane resin and from 3 to 7 parts by mass of silica.

2. The lens for ultrasonic diagnosis apparatuses according to claim 1, wherein the lens has a mass change from 0.01 to 0.2% measured with the following method:
<Method of Measuring a Mass Change>
(1) Prepare a test piece in the shape of a disc with a diameter of 125 mm and a thickness of 2.015 mm, having a silicone rubber layer and a urethane coat layer placed on the silicone rubber layer, wherein materials for the test piece are the same as those for the lens for ultrasonic diagnosis apparatuses. Mount the test piece on a fixing table of an abrasion tester according to JIS K6264-2, and fix it to the table.
(2) Fit two disc-shaped grindstones with a circumference of 160 mm and a thickness of 13 mm to shafts of the abrasion tester, so that a common axis thereof is perpendicular to a plane including the axis of the test piece, and a distance between a first face of one grindstone and a second face of the other grindstone is 52 mm, wherein the first face is opposite the second face with the plane as center plane in between.
(3) Press the grindstones to the test piece so that a contact face between a grinding surface of each grindstone and the disc-shaped test piece is 1 mm by 13 mm in dimensions. Make the fixing table 3000 turns at a rotational speed of 60 r.p.m., with keeping the grindstones pressed to the test piece. Meanwhile the grindstones are rotated by the turns of the disc-shaped test piece.
(4) Measure an initial mass of the test piece before it is fixed to the fixing table and a final mass of the test piece after 3000 turns. Calculate the difference between the initial mass and the final mass. The mass change (%) is defined as the ratio of the difference to the initial mass in percentage terms.

3. The lens for ultrasonic diagnosis apparatuses according to claim 1, wherein the urethane coat layer is made by applying a urethane resin composition including 100 parts by mass of the urethane resin and from 3 to 7 parts by mass of the silica, to the outer surface of the base and curing the applied urethane resin composition.

4. The lens for ultrasonic diagnosis apparatuses according to claim 1, wherein the urethane coat layer preferably has a thickness from 10 to 50 μm.

5. A probe for ultrasonic diagnosis apparatuses comprising:
a case having at least one open end;
the lens for ultrasonic diagnosis apparatuses according to claim 1, arranged so that a tip of the lens is exposed at the open end;
a piezoelectric element housed in the case;
an acoustic matching layer arranged between the lens and the piezoelectric element; and
a backing layer disposed opposite the acoustic matching layer with the piezoelectric element in between.

6. The lens for ultrasonic diagnosis apparatuses according to claim 2, wherein the urethane coat layer preferably has a thickness from 10 to 50 μm.

7. A probe for ultrasonic diagnosis apparatuses comprising:
a case having at least one open end;
the lens for ultrasonic diagnosis apparatuses according to claim 2, arranged so that a tip of the lens is exposed at the open end;
a piezoelectric element housed in the case;
an acoustic matching layer arranged between the lens and the piezoelectric element; and
a backing layer disposed opposite the acoustic matching layer with the piezoelectric element in between.

8. A probe for ultrasonic diagnosis apparatuses comprising:
a case having at least one open end;
the lens for ultrasonic diagnosis apparatuses according to claim 4, arranged so that a tip of the lens is exposed at the open end;
a piezoelectric element housed in the case;
an acoustic matching layer arranged between the lens and the piezoelectric element; and
a backing layer disposed opposite the acoustic matching layer with the piezoelectric element in between.

9. A probe for ultrasonic diagnosis apparatuses comprising:
a case having at least one open end;
the lens for ultrasonic diagnosis apparatuses according to claim 6, arranged so that a tip of the lens is exposed at the open end;
a piezoelectric element housed in the case;
an acoustic matching layer arranged between the lens and the piezoelectric element; and
a backing layer disposed opposite the acoustic matching layer with the piezoelectric element in between.
